(19) United States
(12) Patent Application Publication
(10) Pub. No.: US 2008/0234552 A1
(43) Pub. Date: Sep. 25, 2008

(54) BMI Ruler

(75) Inventor: Vladimir Averbach, Suffern, NY (US)

Correspondence Address: VLADIMIR AVERBACH, 46 MILFORD LANE, SUFFERN, NY, 10901, US

(73) Assignee: Vladimir Averbach, Suffern, NY (US)

(21) Appl. No.: 11/689,413

(22) Filed: Mar. 21, 2007

(51) Int. Cl. A61B 5/00

(52) U.S. Cl. 600/300

(57) ABSTRACT

A device for measuring the Body Mass Index (BMI) of an adult, providing a measure of weight for height. The device allows for an easy and quick determination of BMI or the adult weight status. The device consists of a hand held slide ruler instrument on which a combination of an adult's weight and height immediately indicates the corresponding BMI. Using this device, an adult over 18 years old can determine how much weight he/she would need to lose or gain in order to achieve normal weight, as specified by the current US government guidelines.

### FIELD OF INVENTION

This invention relates to measuring instruments and more specifically to slide rulers. This invention relates to various other tools for aiding computations of moderately complex mathematical formulas, useful for different purposes. In particular this invention utilizes a well known formula for calculating Body Mass Index (BMI).

### BACKGROUND OF THE INVENTION

The Body Mass Index (BMI) is a measure of the weight of a person according to height. It is used as a simple means of classifying individuals according to their body fat content.

A frequent use of the BMI is to assess how much an individual's body weight departs from what is normal or desirable for a person of his/her height. The weight excess or deficiency may, in part, be accounted for by body fat, although other factors such as muscularity also affect BMI.

Human bodies rank along the index from around 15 (near starvation) to over 40 (morbidly obese). This statistical spread is usually described using categories: severe underweight, underweight, optimum weight, pre-obese (or overweight), obese, morbidly obese. The exact index values used to determine weight categories vary from authority to authority, but in general a BMI less than 18.5 is underweight and may indicate malnutrition, an eating disorder, or other health problems, while a BMI greater than 25 is overweight and above 30 is considered obese. These range boundaries apply to adults over 18 years of age.

BMI Categories do not take into account factors such as frame size and muscularity. Despite this, BMI categories are generally regarded as a satisfactory tool for measuring sedentary individuals. It has been used by the World Health Organization (WHO) as the standard for recording obesity statistics since the early 1980s. The BMI ranges are based on the effect body weight has on disease and death. As BMI increases, the risk for some diseases increases. Some common conditions related to overweight and obesity include: premature death, cardiovascular disease, high blood pressure, osteoarthritis, some cancers, diabetes. BMI is only one of many factors used to predict risk for disease. BMI cannot be used to tell a person if he/she has a disease such as diabetes or cancer. It is important to remember that weight is only one factor that is related to disease.

The BMI is calculated from an individual's weight and height as follows: BMI=703 times weight divided by height squared where weight=the weight of the person in lbs and height=height of the person in inches or BMI=weight divided by height squared where weight=the weight of the person in kilograms and height=height of the person in meters.

The mass availability of a simple BMI tool which is user-friendly, small in size, and provides a virtually instantaneous calculation, would make checking person's own BMI progress towards a healthier standard easy and commonplace.

### SUMMARY OF THE INVENTION

The present invention provides a device for calculating the Body Mass Index (BMI) which is user-friendly, small in size, and provides a virtually instantaneous calculation of the BMI based upon the BMI formula. The device of the invention would be particularly useful for all sedentary adults over the age of 18 who are interested in getting their weight closer to the recommended healthy range.

The preferred embodiment of the device comprises a hand-held precision sliding ruler instrument designed to provide person's BMI by utilizing person's weight and height measurements. In the preferred embodiment the device can give results based on both metric and imperial styles of measurement on either side of the instrument.

The device of the invention is thus well suited for adults interested in meeting the guidelines for the normal weight, and also for healthcare professionals who could also hand this tool out to their patients to provide a simple check of the guidelines and encourage healthier eating habits. The device's small size, ease of use, and almost instantaneous results encourage continued use and promote adherence to health guidelines.

The present invention therefore provides a device for calculating a Body Mass Index of an adult, comprising a wholly planar frame, an internal slide disposed within the frame in sliding relation, slidable in a longitudinal direction through the frame, first and second sets of linear mating edges disposed between the frame and the internal slide, the first set of mating edges providing a first mating edge comprising a height scale representing a logarithmic equivalent to a height of the adult and a second mating edge comprising a weight scale representing a logarithmic equivalent to a weight of the adult, the second set of mating edges providing a third mating edge comprising an arrow pointing to a fourth mating edge comprising a BMI scale representing a logarithmic equivalent to a Body Mass Index of the adult, whereby when the height of the adult in the height scale is aligned with weight of the patient in the weight scale, the arrow is aligned with the Body Mass Index of the adult in the BMI scale.

Further describing the invention: the internal slide comprises tongues slidably received in grooves in the frame; the frame and internal slide have opposite faces, the scales on the opposite faces being adapted to calculate the Body Mass Index in two possible styles of measurement: metric and imperial.

### BRIEF DESCRIPTION OF THE DRAWINGS

In drawings which illustrate by way of example only a preferred embodiment of the invention,

FIG. 1 is a front elevation of a device embodying the invention showing scales for calculating BMI of an adult person using the imperial units of measurement,

FIG. 2 is a rear elevation of a device of FIG. 1 showing scales for calculating BMI of an adult person using the metric units of measurement,

FIG. 3 is a cross-sectional 3-D perspective view of the device of FIG. 1,

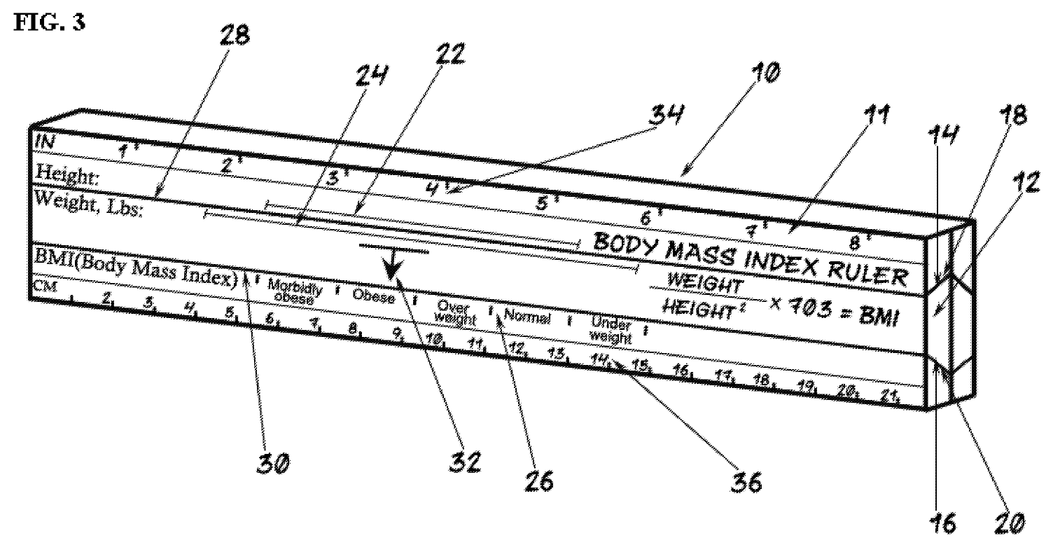

FIG. 4 is a front elevation of the device in FIG. 1 showing the computer calculated scales for calculating BMI of an adult person using the imperial units of measurement.

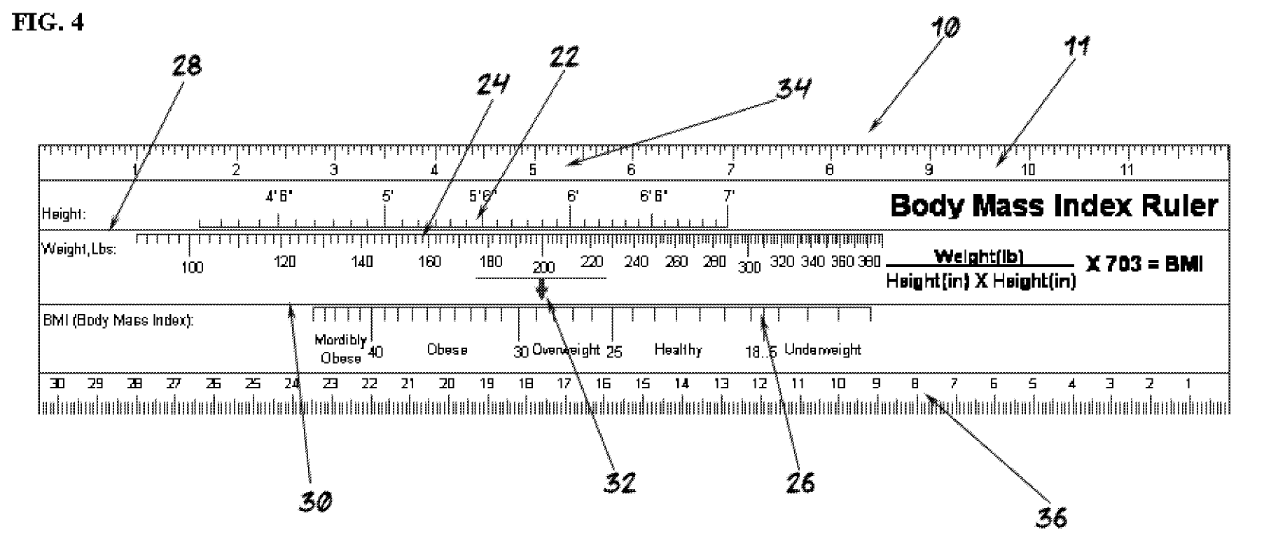

FIG. 5 is a rear elevation of the device in FIG. 4 showing the computer calculated scales for calculating BMI of an adult person using the imperial units of measurement.

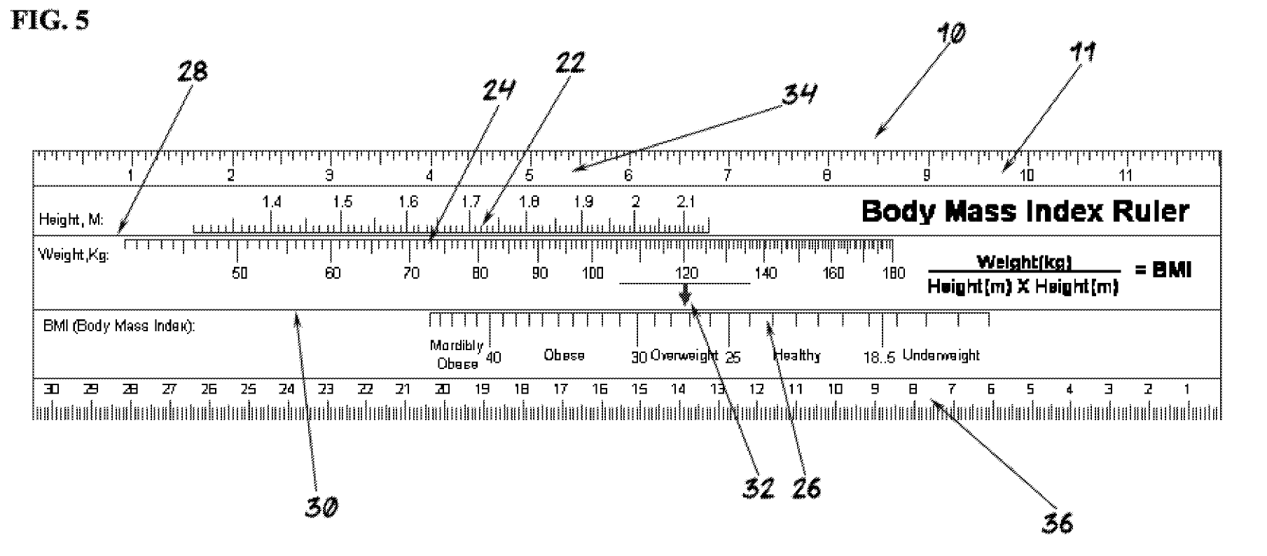

### DETAILED DESCRIPTION OF THE INVENTION

As noted above, the formula which is used to determine the Body Mass Index of an adult is as follows: BMI=703 times (weight/height squared)

in which

weight=the weight of the person in pounds, and

height=the height of the person in inches.

This formula can be adapted to metric units where it becomes: BMI=weight/height squared

in which

weight=the weight of the person in kilograms, and

height=the height of the person in meters.

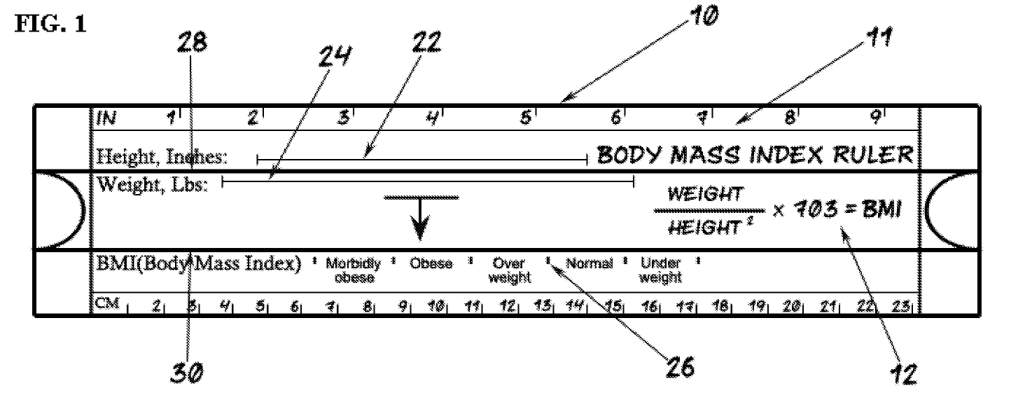

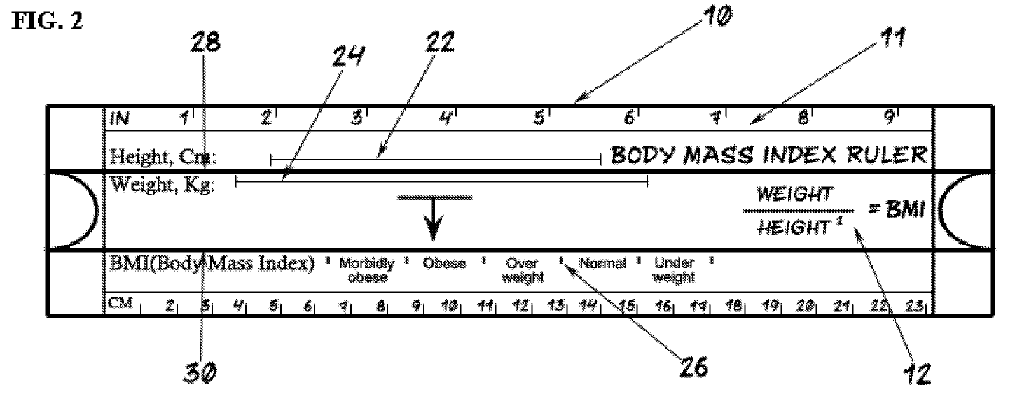

FIGS. 1 and 2 illustrate a first preferred embodiment of the invention for solving the BMI according to the above formula on an individual-specific basis (description of the ruler from the figure follows).

To solve for BMI requires two arithmetic operations, a multiplication and a division, and operates over a restricted range of values for each variable. The range for each variable can be set out in finite scales 22, 24, 26 disposed along the set of mating edges 28, 30 of the internal slide.

In order to perform multiplication operations (i.e. one multiplication and one division) to solve the BMI formula, the multiplication operations are converted to addition operations by use of logarithmic functions, specifically: log(a.times.b)=log(a)+log(b)

Also: log(a/b)=log(a)−log(b)

Then, using logarithms it is possible to multiple variables in the BMI formula by adding corresponding logarithmic values. This is achieved by plotting the ranges of the two variables onto logarithmic scales 22, 24, 26 Adding the distances represented by the logarithmic scales is equivalent to adding the logarithms of the values, and thus multiplying the values.

The height scale 22 is disposed on the frame 11 along the first mating edge 28, in opposition to the weight scale 24 which is disposed along the internal slide 12. The BMI indicator arrow 32 is disposed on the internal slide 12 along the second set of mating edges 30 in opposition to the BMI scale 26. For each variable, the scales are printed in such a way that the BMI result in the BMI scale 26 according to the BMI formula is at the correct location on the frame 11 relative to the BMI indicator arrow 32 when the applicable values in the height scale 22 and weight scale 24 are aligned along the first set of mating edges 28.

In operation, an individual aligns the weight value in the weight scale 24 against the height value in the height scale 22 The value on the BMI scale 26 opposite to the BMI indicator arrow 32 provides the individual's Body Mass Index.

In the preferred embodiment the device 10 can provide either metric units of height (m) and weight (kg), as in the embodiment of FIG. 2, or imperial units of height (in) and weight (lbs), as in the embodiment of FIG. 1. The device 10 may be constructed from plastic or any other suitable material, with machine printed scales on both faces of the device 10 to provide calculations for individuals preferring either metric or imperial units of measurement, using a single device 10. Calibration of the scales 22, 24, 26 can be achieved by a computer program, which converts the scales 22, 24, 26 to logarithmic equivalents and aligns the scales.

The preferred embodiment of the present invention having been thus described in detail by way of example, it will be apparent to those skilled in the art that variations and modifications may be made without departing from the invention. The invention includes all such variations and modifications as fall within the scope of the appended claims.

1. A device for calculating a Body Mass Index (BMI) of an adult, comprising a substantially planar frame, an internal slide disposed within the frame in sliding relation, slidable in a longitudinal direction through the frame, first and second sets of liner mating edges disposed between the frame and the internal slide, the first set of mating edges providing a first mating edge comprising a height scale representing a logarithmic equivalent to a height of the adult and a second mating edge comprising a weight scale representing a logarithmic equivalent to a weight of the adult, the second set of mating edges providing a third mating edge comprising an arrow pointing to a fourth mating edge comprising a BMI scale representing a logarithmic equivalent to a Body Mass Index of the adult, whereby when the height of the adult in the height scale is aligned with the weight of the patient in the weight scale, the arrow is aligned with the Body Mass Index of the adult in the BMI scale.

2. The device of claim 1 in which the internal slide comprises tongues sidably received in grooves in the frame.

3. The device of claim 2 in which the internal slide comprises an arrow to assist in visually aligning values on the scales.

4. The device of claim 1 in which the frame and internal slide have opposite faces, the scales on the opposite faces being adapted to calculate the Body Mass Index in two possible styles of measurement: metric and imperial.

5. The device of claim 1 in which the scales are calculated by a computer and printed on the device.
